(19) United States
(12) Patent Application Publication
(10) Pub. No.: US 2025/0318713 A1
(43) Pub. Date: Oct. 16, 2025

(54) COOLANT DELIVERY DEVICE

(71) Applicant: ENDOCISION TECHNOLOGIES INC., Montreal (CA)

(72) Inventors: Marc CHELALA, Montreal (CA); Bruno CHABOT, Montreal (CA); Iman KIA, Montreal (CA); Daniel TASSE, Montreal (CA); Carl KHAIRALLAH, Montreal (CA); Gilbert MILANOVIC, Montreal (CA); Abraham Moishe LIBERMAN, Montreal (CA); Andre TREMBLAY, Montreal (CA)

(21) Appl. No.: 19/176,299

(22) Filed: Apr. 11, 2025

Related U.S. Application Data
(60) Provisional application No. 63/633,373, filed on Apr. 12, 2024.

(51) Int. Cl. A61B 1/00; A61B 18/00; A61B 18/02

(52) U.S. Cl. CPC A61B 1/0014; A61B 1/00042; A61B 18/02; A61B 2018/00172; A61B 2018/0212

(57) ABSTRACT

A coolant delivery device configured to be attachable to an endoscope, the coolant delivery device including a coolant component and an attachment mechanism. The coolant component having a coolant component body configured to removably connect to a coolant source, the coolant source containing a coolant. The coolant component body defining a fluid path from the coolant source to an outlet of the coolant component body. The coolant component includes an actuation mechanism operatively coupled to the coolant component body and configured to control flow of the coolant from the coolant source. The attachment mechanism configured to removably attach the coolant component to the endoscope.

### CROSS REFERENCE

The present application claims priority from U.S. Provisional Patent Application No. 63/633,373, filed on Apr. 12, 2024, the entirety of which is incorporated by reference herein.

### TECHNICAL FIELD

The present technology relates to coolant delivery devices, specifically a coolant delivery device which may be configured to be attachable to an endoscope.

### BACKGROUND

Cryotherapy is based on the principle that short applications of extreme cold can enable tissue removal and/or produce localized tissue destruction. In surgical procedures, cryoprobes use a coolant, such as carbon dioxide, nitrogen, or argon, for the purpose of tissue adhesion (e.g., to remove tissue samples, biopsies, blood clots, mucus plugs, etc.) and/or freezing and destroying tissue. Conventional cryoprobes include a closed tip and deliver the coolant via a catheter, such as a delivery tube, to the closed tip where the coolant rapidly vaporizes. As the liquid coolant vaporizes, heat from the cryoprobe tip is absorbed, lowering the temperature at the tip. Temperatures of tissues in contact with the tip are subsequently lowered, thereby freezing and destroying the tissue.

Cryoprobes are often used in conjunction with endoscopes to guide the cryoprobe to the target site. Endoscopes are a thin, flexible tube, which include a light source and a camera to allow the user to view images of the target site on a monitor. Endoscopes (such as bronchoscopes, cystoscopes, duodenoscopes, endobronchial ultrasound, endoscopic ultrasound, etc.) include a control handle to enable the user to steer and adjust the tip of the endoscope.

Typically, in surgical procedures, cryoprobes are connected to a source of the coolant stored in pressurized tanks, via a supply line. These pressurized tanks take up a large footprint within the operating room and are not easily portable, requiring a trolley to move. Delivery of the coolant from the coolant source to the cryoprobe is controlled via an electromechanical control unit which enables the user to adjust various parameters such as intensity and duration of the flow of the coolant, tailoring the parameters to the specific medical procedure and target tissues. The control unit of the cryoprobe is separate from the control handle of the endoscope and may be controlled via a foot pedal, the user's other hand, or by another person. Thus, user's control the control unit to delivery fluid via the electromechanical control unit while separately controlling the endoscope via the control handle.

There is thus a desire to develop a coolant delivery device which overcomes the inconveniences present in the prior art.

### SUMMARY

It is an object of the present technology to ameliorate at least some of the inconveniences present in the prior art.

Broadly, there is provided a coolant delivery device which utilizes a cartridge of coolant and may be controlled by single-handedly by a single person, thereby providing a portable solution, minimizing a footprint and reducing a cost by avoiding the need for large, pressurized tanks of coolant and a separate control unit for the cryoprobe. The coolant delivery device of the present technology is attachable to an endoscope which, in some instances, may enable the user to control the endoscope and deliver coolant using a single hand. a

According to one aspect of the present technology, there is provided a coolant delivery device configured to be attachable to an endoscope, a coolant component having: a coolant component body configured to removably connect to a coolant source, the coolant source containing a coolant, the coolant component body defining a fluid path from the coolant source to an outlet of the coolant component body; and an actuation mechanism operatively coupled to the coolant component body and configured to control flow of the coolant from the coolant source; and an attachment mechanism configured to removably attach the coolant component to the endoscope.

In some embodiments, the attachment mechanism is an endoscope component connected to the coolant component body, the endoscope component defining a channel for selectively receiving a portion of the endoscope.

In some embodiments, the endoscope component includes a sleeve portion configured to wrap at least partially around the portion of the endoscope, the sleeve portion defining the channel in which the portion of the endoscope can be received to extend through open ends of the sleeve portion, the sleeve portion being longitudinally split to define a channel opening through which the portion of the endoscope will be received and removed.

In some embodiments, the coolant delivery device further includes a securing mechanism operatively connected to the endoscope component and configured to modulate a width of the channel opening for adjustably securing the endoscope component to the endoscope.

In some embodiments, the securing mechanism is a clip pivotably connected to the endoscope component, the clip configured to adjust a tension of the attachment of the endoscope component onto the portion of the endoscope.

In some embodiments, the endoscope component includes a pair of wings, a first of the pair of wings extending from a first edge of the sleeve portion defining the channel opening, and a second of the pair of wings extending from a second edge of the sleeve portion defining the channel opening; and the clip being pivotably connected to the first of the pair of wings and having a free end configured to connect to the second of the pair of wings at different positions for adjusting the width of the channel opening.

In some embodiments, the coolant component body is disposed on the sleeve portion of the endoscope component.

In some embodiments, the endoscope component and the coolant component body are formed as a unitary piece.

In some embodiments, the endoscope component and the coolant component are removably connected to one another.

In some embodiments, the coolant component body includes a first engaging member; the endoscope component includes a second engaging member; and the first engaging member is removably connected to the second engaging member.

In some embodiments, the first engaging member is a guide extending away from the coolant component body; and the second engaging member is a slot, the guide being slidably received in the slot.

In some embodiments, the slot includes a dovetail-shaped profile and the guide includes a complementary dovetail-shaped profile.

In some embodiments, the guide includes a snap clip disposed at one end of the guide to releasably secure the guide into the slot.

In some embodiments, the channel of the endoscope component is configured to receive at least one of a lower portion of a control handle of the endoscope and an instrument port of the endoscope.

In some embodiments, the endoscope component extends laterally from the coolant component body.

In some embodiments, the endoscope component defines a notch configured to receive a cable of the endoscope.

In some embodiments, the endoscope component is composed of a resilient material.

In some embodiments, the channel has an inner surface; and the endoscope component further includes a liner disposed on at least a portion of the inner surface of the channel.

In some embodiments, the coolant component body further includes a valve operably connected to the actuation mechanism for controlling flow of the coolant.

In some embodiments, the coolant component further includes a cover removably connected to the coolant component body and configured to house the coolant source.

In some embodiments, a catheter having an exhaust hub disposed at a proximal end of the catheter, the exhaust hub being connectable to the outlet of the coolant component body to fluidly connect the catheter and the outlet.

In some embodiments, the exhaust hub has a first end connectable to the outlet, and a second end connectable to the catheter; and the exhaust hub defines: a coolant channel extending therebetween and in fluid communication with the outlet and the catheter, when the exhaust hub is connected to the outlet and the catheter; and a plurality of exhaust channels extending therebetween and in fluid communication with the catheter, each of the plurality of exhaust channels being disposed radially around the coolant fluid channel for expelling exhaust gas.

In some embodiments, the actuation mechanism is a lever pivotably connected to the coolant component body.

In some embodiments, the lever includes a pivot end and a free end; the pivot end is pivotably connected to the coolant component body; and the free end of the lever is disposed upwardly of the pivot end when the coolant delivery device is attached to the endoscope.

In some embodiments, the lever is pivotably movable between: an unactuated position; and an actuated position in which the lever is pivoted towards the coolant component body to dispense the coolant from the coolant source.

In some embodiments, the coolant component defines a coolant component longitudinal plane extending through a central axis of the coolant component; the endoscope component defines an endoscope component longitudinal plane extending through a central axis of the endoscope component; and the coolant component longitudinal plane and the endoscope component longitudinal plane are not parallel.

In some embodiments, an angle between the coolant component longitudinal plane and the endoscope component longitudinal plane is between 30° and 60°.

In some embodiments, the lever includes a grip portion extending from a side edge of the lever.

In some embodiments, the grip portion is disposed at a distal end of the lever.

In some embodiments, the lever is pivotably connected to a lower section of the coolant component body; and the grip portion is disposed upwardly from the lower section of the coolant component body.

In some embodiments, the coolant delivery device further includes the endoscope, and the endoscope has a front end and a back end, the front end configured for a user's fingers to rest; and the back end configured for a user's thumb to rest; and the lever being disposed towards the front end of the endoscope when the coolant delivery device is attached to the endoscope such that the lever can be actuated by at least one of the user's fingers.

In some embodiments, the coolant delivery device further includes the endoscope.

In some embodiments, the coolant delivery further includes the coolant source.

Another broad aspect of the present technology provides a kit including the coolant delivery device and the coolant source.

Another abroad aspect of the present technology provides a coolant component of a coolant delivery device. The coolant component includes a coolant component body configured to removably connect to a coolant source, the coolant source containing a coolant; and a lever pivotably connected to the coolant component body for controlling flow of the coolant, the lever being selectively pivotable between: an unactuated position; and an actuated position in which the lever is pivoted towards the coolant component body, in response to the lever being in the actuated position, the coolant is dispensed from the coolant source through an outlet of the coolant component body.

Another broad aspect of the present technology provides an exhaust hub connectable to a catheter and a coolant delivery device. The hub includes a first end and a second end; the hub defining: a coolant channel extending between the first and the second end, the coolant channel being configured to fluidly connect the catheter and the coolant delivery device; and a plurality of exhaust channels configured to fluidly connect to the catheter, each of the plurality of exhaust channels extending between the first and the second end and positioned radially around the coolant channel.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of embodiments of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a perspective view taken from a front side of a coolant delivery device and including a user's hand, according to non-limiting embodiments of the present technology;

FIG. 2 depicts a top plan view of the coolant delivery device of FIG. 1;

FIG. 3 depicts a perspective view taken from the front side of the coolant delivery device of FIG. 1, with the user's hand removed;

FIG. 4 depicts a perspective view from a rear side of the coolant delivery device of FIG. 3;

FIG. 5 depicts a perspective view taken from the front side of the coolant delivery device of FIG. 3;

FIG. 6 depicts a front plan view of the coolant delivery device of FIG. 3;

FIG. 7 depicts a perspective view taken from the front side of the coolant delivery device of FIG. 3, with a coolant component removed;

FIG. 8 depicts a side view of a coolant component and an exhaust hub of the coolant delivery device with an endoscope component and endoscope removed;

FIG. 9 depicts a side view of the exhaust hub of FIG. 8;

FIG. 10 depicts a top end view of the exhaust hub of FIG. 8;

FIG. 11 depicts a side view of the exhaust hub of FIG. 8, with a catheter removed;

FIG. 12 depicts a cross-sectional view of the exhaust hub of FIG. 8, taken along line A-A of FIG. 11;

FIG. 13 depicts a method of assembling and using the coolant delivery device of FIG. 1;

FIG. 14 depicts a perspective view of positioning the endoscope component of the coolant delivery device of FIG. 1 onto the endoscope;

FIG. 15 depicts a perspective view of securing the endoscope component of the coolant delivery device of FIG. 1 onto the endoscope;

FIG. 16 depicts a perspective view of connecting a coolant source onto the coolant component of the coolant delivery device of FIG. 1;

FIG. 17 depicts a perspective view of connecting a cover onto the coolant component of the coolant delivery device of FIG. 1;

FIG. 18 depicts a perspective view of connecting the exhaust hub onto the coolant component of the coolant delivery device of FIG. 1;

FIG. 19 depicts a perspective view of connecting the coolant component onto the endoscope component of the coolant delivery device of FIG. 1; and

FIG. 20 depicts a perspective view of a user actuating a lever of the coolant component of the coolant delivery device to dispense coolant.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, “having”, “containing”, “involving” and variations thereof herein, is meant to encompass the items listed thereafter as well as, optionally, additional items. In the following description, the same numerical references refer to similar elements.

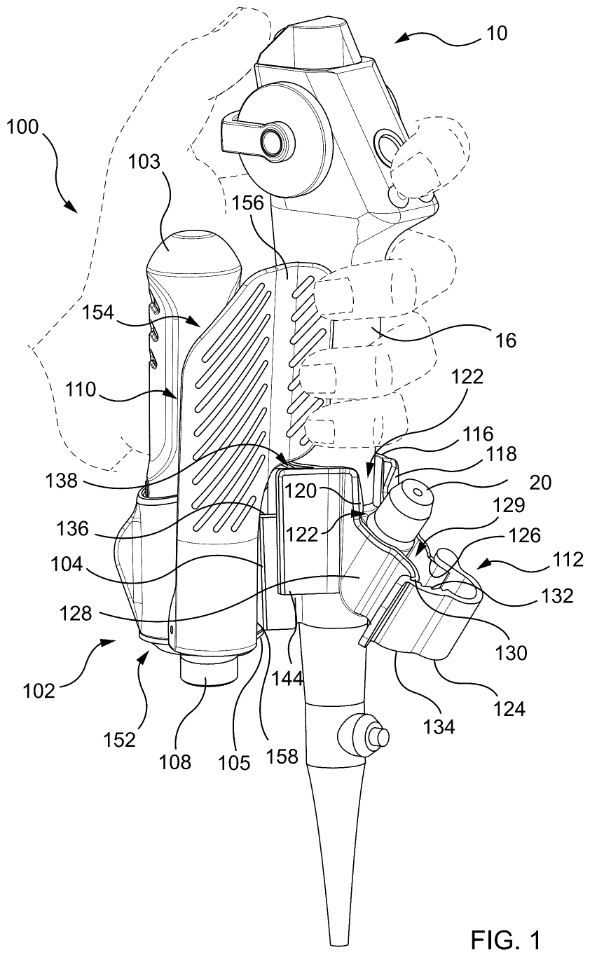

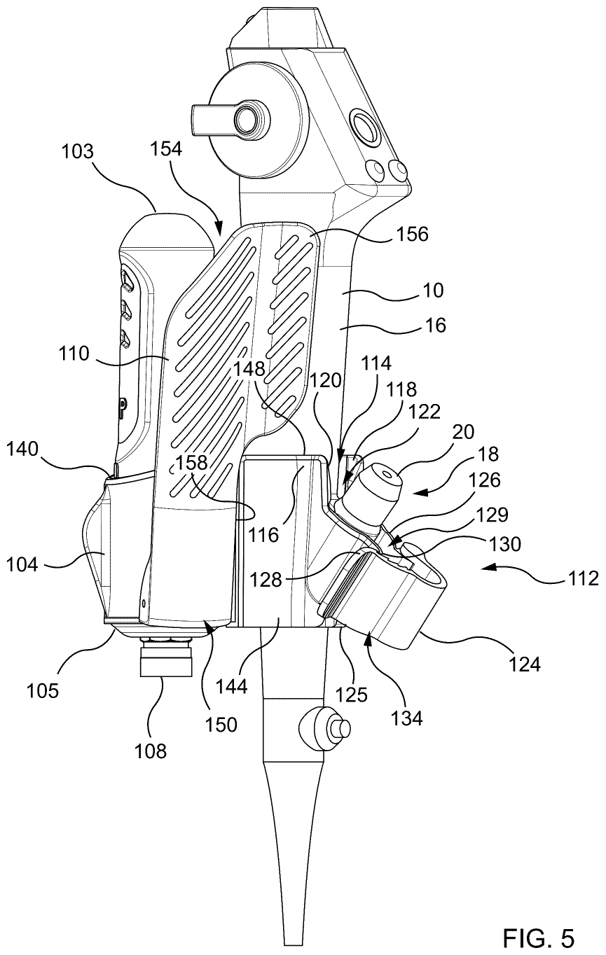

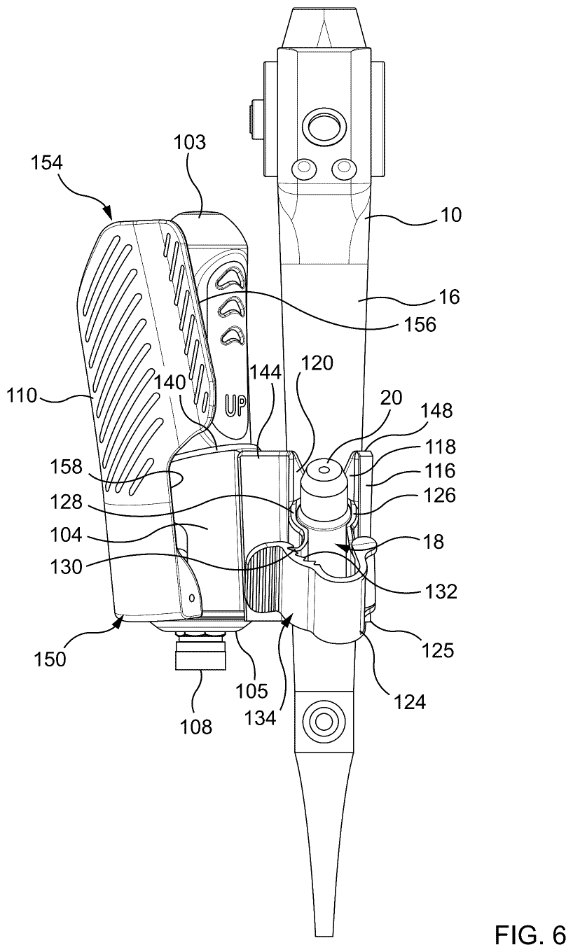

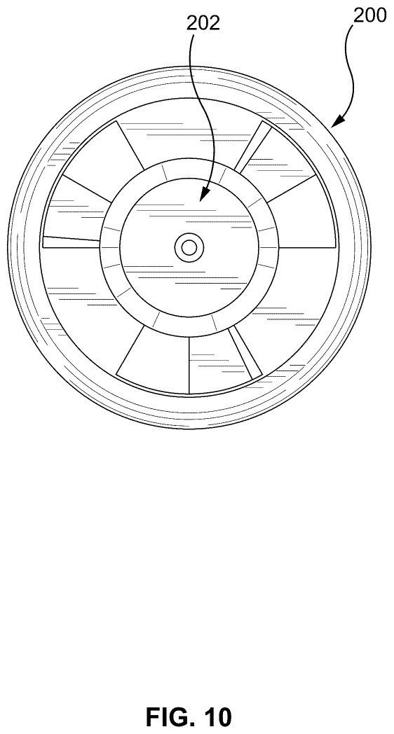

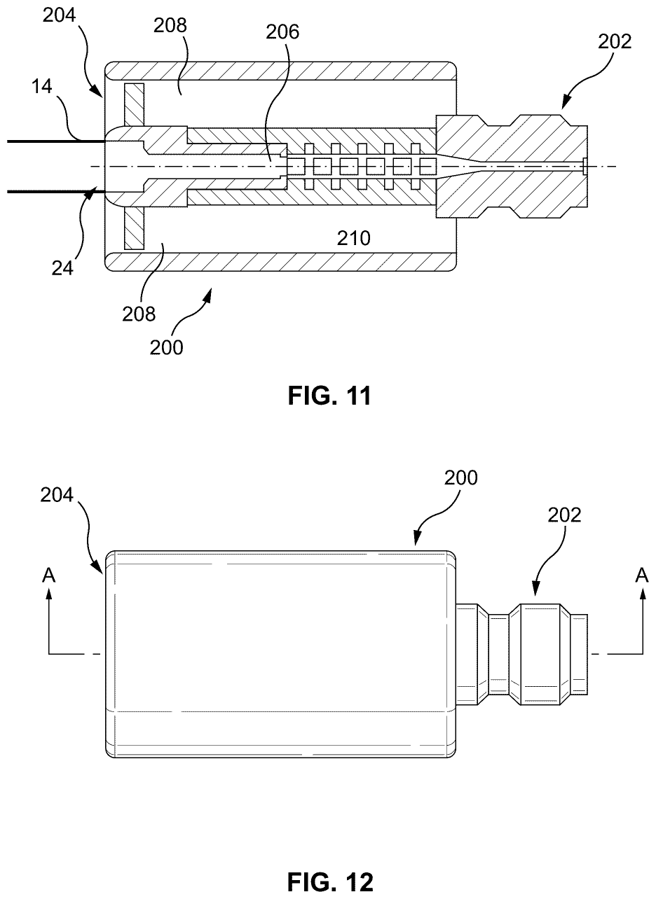

FIGS. 1 to 12 and 14 to 20 depict an embodiment of a coolant delivery device 100 of the present technology. The coolant delivery device 100 is configured to be removably attachable to an endoscope 10, such that a user may easily attach and remove the coolant deliver device 100 from the endoscope 10.

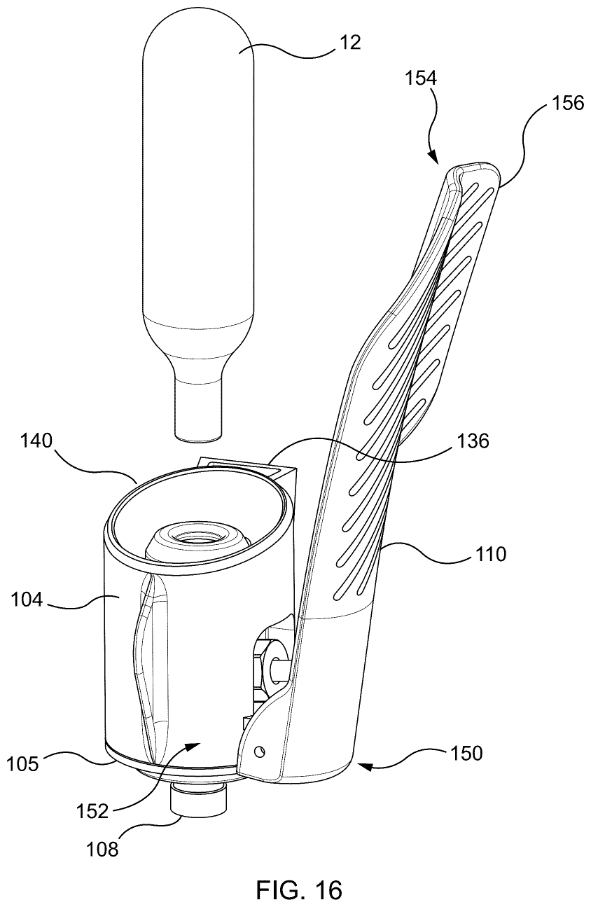

The coolant delivery device 100 includes a coolant component 102 having a coolant component body 104 configured to removably connect to a coolant source 12. In this embodiment, the coolant source 12 is a disposable cartridge containing a pressurized coolant (depicted in FIG. 16). In some embodiments, the coolant may be one or more of carbon dioxide, nitrogen gas, or argon gas. It is contemplated that, in alternative embodiments, another coolant may be contained within the cartridge. The coolant source 12 cartridge is formed of metal. In certain embodiments, for single-use cartridges, the cartridge may be welded or crimped shut. In this instance, when connecting the cartridge to the coolant component body 104, the welded or crimped end is punctured. Alternatively, in re-sealable cartridges, the cartridge may include a valve at the end. In this case, when connecting the cartridge to the coolant component body 104, the coolant component body 104 pushes the valve inward. It is further contemplated that the coolant source may be a pressurized tank filled with coolant and connected via a supply line.

In this embodiment, the coolant delivery device 100 further includes a cover 103 for housing the coolant source 12. The cover 103 is positioned overtop of the coolant source 12 and removably connects to the coolant component body 104 via a threaded interface. The cover 103 houses the coolant source 12 such that, during use, the user does not contact the cold, pressurized cartridge. It is to be understood that the cover 103 may be connected to the coolant component body 104 by alternative connecting mechanisms in different embodiments. It is contemplated that, in other embodiments, the cover 103 may be omitted.

An outlet 106 is defined in a bottom face 105 of the coolant component body 104. The outlet 106 is in fluid communication with the coolant source 12. In other words, the coolant component body 104 defines a fluid path between the coolant source 12 and the outlet 106 such that coolant may flow from the coolant source 12 through the outlet 106. In this embodiment, the outlet 106 includes a quick-connect mechanism 108 for coupling the coolant delivery device 100 to a hub 200 of a catheter 14. The quick-connect mechanism 108 fluidly connects the catheter 14 to the coolant source 12 such that coolant is deliverable to a distal tip of the catheter 14 (not shown) for freezing the target tissue. The hub 200 and catheter 14 will be described in further detail below.

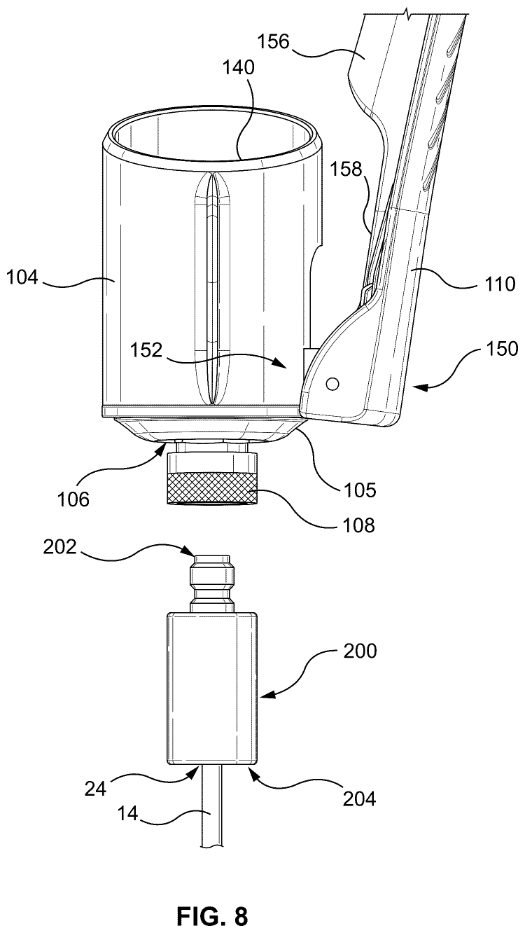

An actuation mechanism 110 is operatively coupled to the coolant component body 104. The actuation mechanism 110 controls the flow of coolant from the coolant source 12 to the outlet 106 of the coolant component body 104. In this embodiment, the actuation mechanism 110 is a mechanical lever 110 operatively connected to a valve (not shown) disposed within the coolant component body 104 for dispensing coolant. The lever 110 is pivotably connected to the coolant component body 104. The lever 110 has a pivot end 150 which is pivotably connected to a lower portion 152 of the coolant component body 104 (as shown in FIG. 8). The lever 110 has a free end 154 disposed upwardly from the pivot end 150. The user interacts with the free end 154 of the lever 110 to control the flow of coolant from the coolant source 12.

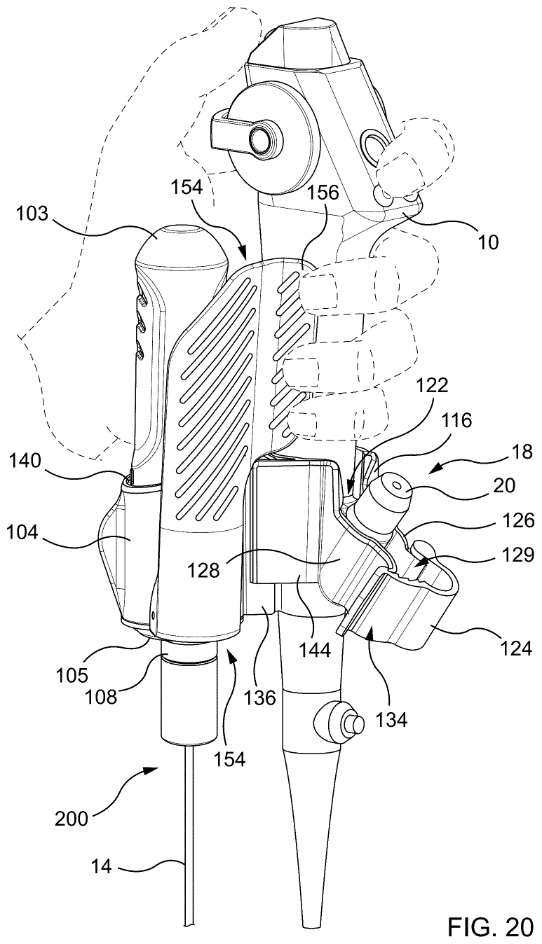

A grip portion 156 is positioned at the free end 154 of the lever 110 to provide the user with a gripping surface that can be reached by the user's fingers while the user is holding the endoscope 10 (as shown in FIG. 20). The grip portion 156 extends away from a side edge 158 of the lever 110. The grip portion 156 extends longitudinally down the side edge 158.

As described above, the lever 110 is operably connected to the valve (not shown) disposed within the coolant component body 104 to control the flow of coolant from the coolant source 12. The lever 110 is movable between an unactuated position, in which the valve is closed so that there is no flow of coolant, and an actuated position, in which the valve is opened and there is flow of coolant. The lever 110 is resiliently biased to the unactuated position. The lever 110 is movable towards the coolant component body 104, such that in the actuated position, the lever 110 is closer to the coolant component body 104.

It is appreciated that the configuration of the actuation mechanism 108 may vary in different embodiments, for example the actuation mechanism 108 may be a button or a pull tag. Alternatively, the actuation mechanism 108 may be an electromechanical system which controls flow of the coolant, monitors flow of the coolant, and/or may include an indicator or alarm in communication with a user interface. In this instance, actuation of the electromechanical system may be achieved via a finger/hand trigger or a foot pedal.

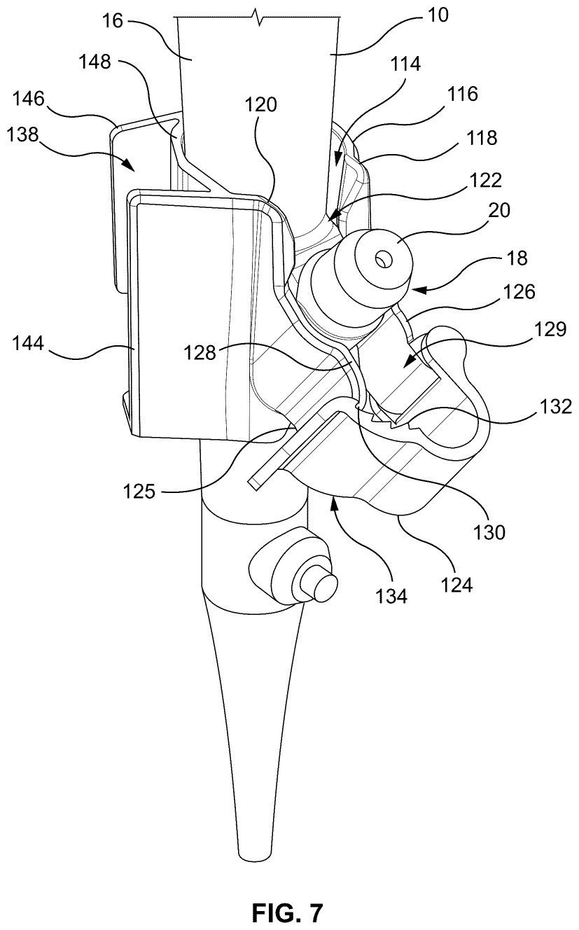

The coolant delivery device 100 further includes an attachment mechanism 112 for removably attaching the coolant component 102 to the endoscope 10. With specific reference to FIG. 7, the attachment mechanism 112 is an endoscope component 112 defining a channel 114 for selectively receiving a portion of the endoscope 10. The endoscope component 112 is designed to avoid interference with a grip section 16 of the endoscope 10, and the user's grip around the endoscope 10. Specifically, the endoscope component 112 fits around a lower portion 18 of the endoscope 10 and a height of the endoscope component 112 does not extend into the grip section 16. It is to be understood that the attachment mechanism 112 may have a different configuration in alternative embodiments to removably attach the coolant component 102 onto the endoscope 10.

In this embodiment, the endoscope component 112 includes a sleeve portion 116 which wraps, at least partially, around the lower portion 18 of the endoscope 10 and defines the channel 114 in which the lower portion 18 of the endoscope 10 is received and partially extends through. The sleeve portion 116 is longitudinally split such that the two ends 118, 120 (a first end 118 and a second end 120) of the sleeve portion 116 define the channel opening 122. The endoscope 10 is inserted into and removed from the sleeve portion 116 via the channel opening 122. It is contemplated that, in alternative embodiments, the channel opening 122 may be omitted. In this instance, for example, the endoscope component 112 may be slid onto the endoscope 10 from a bottom of the endoscope 10, moving the endoscope component 112 upwards. It is contemplated that the attachment mechanism 112 may be configured differently in alternative embodiments to enable the user to removably attach the coolant component 102 to the endoscope 10, for example the attachment mechanism 112 may be a band or a strap which loops around the endoscope 10. It is further contemplated that the sleeve portion 116 may be configured to attach to accessories of a robotic endoscope instead of a handheld endoscope.

The sleeve portion 116 further includes two protrusions 117 (as seen in FIG. 15). Each protrusion 117 is positioned on an upper edge of each of the end 118, 120 of the sleeve portion 116 to prevent the sleeve portion 116 from slipping or moving downwards along the endoscope 10. Specifically, when the sleeve portion 116 is positioned around the lower portion 18 of the endoscope 10, the protrusions 117 secure the sleeve portion 116 against a top of the instrument channel 20. It is contemplated that, in alternative embodiments, the protrusions 117 may be omitted.

The endoscope component 112 includes a pair of wings 126, 128 (a first wing 126 and a second wing 128) which extend from the sleeve portion 116. The first wing 126 extends from an edge of the first end 118 of the sleeve portion 116 and the second wing 128 extends from an edge of the second end 120 of the sleeve portion 116. The pair of wings 126, 128 define a slot 129 extending away from the channel opening 122 of the sleeve portion 116. In this embodiment, the slot 129 is narrower than the channel 114 and receives the lower portion 18 of the endoscope 10 which partially extends out of the channel 114, such as the instrument channel 20 of the endoscope 10.

The endoscope component 112 has a close fit with the endoscope 10. That is, an inner surface of the endoscope component 112 is configured to be substantially flush with an outer surface of the lower portion 18 of the endoscope 10. More specifically, the contours of the channel 114 and an inner surface of the wings 126, 128 match the contours of the outer surface of the lower portion 18 of the endoscope 10. In some embodiments, the endoscope component 112 may be composed of a resilient material, such that as during insertion of the endoscope 10 into the endoscope component 112, the endoscope component 112 flexes outwards to accommodate the insertion, and once the endoscope 10 is received, the endoscope component 112 flexes back to its original shape. In some embodiments, the endoscope component 112 is formed of a polymer, such as acrylonitrile butadiene styrene (ABS) or nylon. In other embodiments, the wall thickness of the endoscope component 112 (that is, the wall thicknesses of the sleeve portion 116 and the wings 126, 128) are selected to enable the endoscope component 112 to flex outwardly during insertion and return to its original shape once the endoscope 10 is received. It is appreciated that an alternative embodiment may have a combination of wall thickness and material to provide flexibility and resilience of the endoscope component 112. In further alternative embodiments, the endoscope component 112 may be rigid but be configured to allow insertion and removal, for example the endoscope component 112 may include a hinge.

It is contemplated that, in alternative embodiments, the endoscope component 112 may only include the sleeve portion 116 and the wings 126, 128 may be omitted.

In some embodiments, a liner may be disposed on at least a portion of the surface of the channel 114 to provide a close fit between the sleeve portion 116 and the endoscope 10. The liner may be composed of a compliant material such that the liner fits to the handle 12 of the endoscope 10 as the sleeve portion 116 is positioned around and secured to (for example, via a securing mechanism described in further detail below) the portion of the endoscope 10. In this instance, the liner enables adaptability of the endoscope component 112 onto various types, sizes, and/or brands of endoscopes 10. That is, the liner compensates for instances where the sleeve portion 116 may be larger than the portion of the endoscope 10. Additionally, the liner may also be made of a material which provides frictional resistance to mitigate rotation of the sleeve portion 116 around the endoscope 10. The liner may be a porous material such as a foam, a sponge, or any other suitable configuration. The liner may be made of any suitable material, such as a polymer.

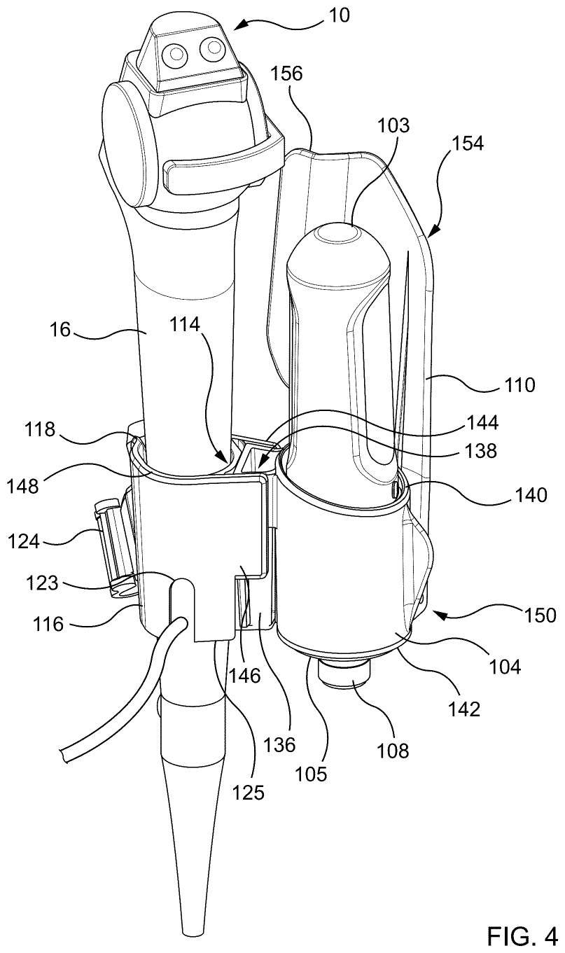

As depicted in FIG. 4, the sleeve portion 116 of the endoscope component 112 defines a notch 123 to accommodate cabling of the endoscope 10. In this embodiment, the notch 123 is defined on a rearward facing part of the sleeve portion 116. The notch 123 extends upwards from a bottom edge 125 of the sleeve portion 116. In alternative embodiments, the notch 128 may instead be an opening or a channel in which the cabling of the endoscope 10 may be threaded through. It is contemplated that, in other embodiments, the notch 123 may be omitted.

A securing mechanism 124 is operatively connected to the endoscope component 112 for securing the endoscope component 112 to the endoscope 10. The securing mechanism 124 provides sufficient securement of the endoscope component 112 to the endoscope 10 such that no counteracting force by the user (i.e., positioning their thumb on the coolant delivery device 100) is required during actuation of the lever 110. Therefore, the user can keep their thumb on the endoscope handle 12 handle to control the endoscope 10 deflection. The securing mechanism 124 is configured to selectively modulate the tension of the endoscope component 112 such that the endoscope component 112 may be adaptable to different configurations and/or sizes of endoscopes 10.

In this embodiment, the securing mechanism 124 is a clip 124 pivotably connected to the sleeve portion 116. Specifically, the clip 124 is pivotably connected to the first wing 126 of the sleeve portion 116. The clip 124 includes a free end 134 that removably secures to the second wing 128. The second wing 128 includes a ridge 130 which engages a notch 132 disposed at the free end 134 of the clip 124, thereby securing the clip 124 in a closed position. The clip 124 is a variable tension clip having a plurality of notches 132 positioned along the free end 134 (as seen in FIG. 15). Each of the notches 132 are engageable with the ridge 130 to enable the user to selectively adjust the tension of the slot 129 defined by the wings 126, 128 and the channel opening 122 around the endoscope 10.

It is contemplated that, in alternative embodiments, the securing mechanism 124 may include different configurations. For example, in one alternative embodiment, the securing mechanism 124 may include a band which is secured around each wing 126, 128. In this example, a certain band size may be selected to adjust the tension of the sleeve portion 116. In another alternative embodiment, the securing mechanism 124 may be a V-channel clip which slides down onto and variably tightens the wings 126, 128.

It is to be understood that, in some embodiments, the securing mechanism 124 is not configured to adjust the tension of the endoscope component 112, rather the securing mechanism 124 enables the securement of the endoscope component 112 to the endoscope 10. It is further contemplated that the securing mechanism 124 may be omitted.

The endoscope component 112 and the coolant component 102 are separate pieces that removably connect to one another via a first engaging member 136 on the coolant component 102 and a second engaging member 138 on the endoscope component 112. It is contemplated that, in alternative embodiments, the coolant component 102 and the endoscope component 112 may be formed as a unitary piece.

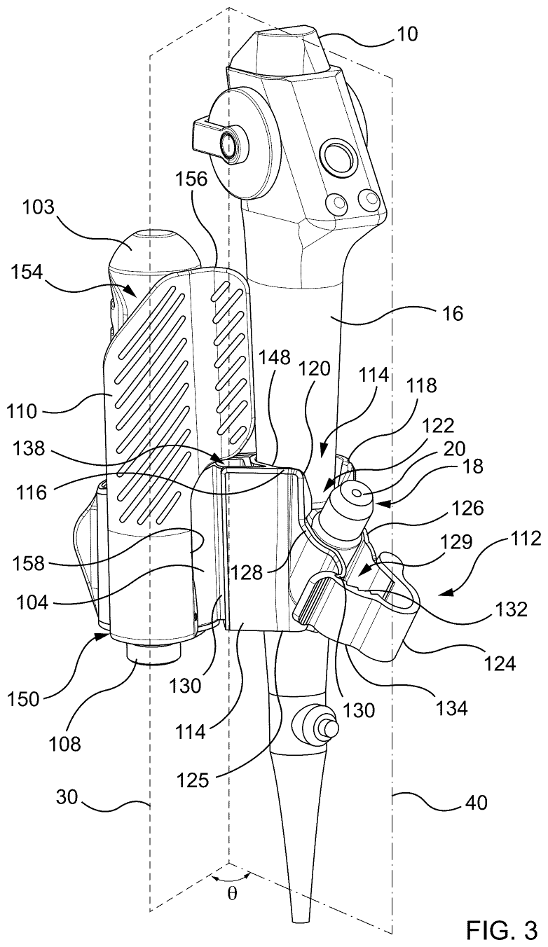

With reference to FIGS. 3, 4, 7, 14 and 19, the first engaging member 136 is a guide 136 disposed on the coolant component body 104 and the second engaging member 138 is a slot 138 configured to slidably receive the guide 136. With specific reference to FIGS. 3, 4, and 19, the guide 136 extends away from an outer surface of the coolant component body 104. The guide 136 extends substantially longitudinally between a top edge 140 and a bottom edge 142 of the coolant component body 104. It is contemplated that, in alternative embodiments, the guide 136 may extend partially between the top edge 140 and the bottom edge 142. The guide 136 has a depth sufficient to provide clearance between the user's fingers (when gripping the endoscope 10) and the cover 103 when the guide 136 is received in the slot 138 and the coolant delivery device 100 is attached to the endoscope 10. In some embodiments, the guide 136 may be hollowed. In alternative embodiments, the guide 136 may be a solid piece.

With continued reference to FIGS. 3, 4, 7, 14, and 19, the sleeve portion 116 of the endoscope component 112 includes a pair of protrusions 144, 146 which define the slot 138. The protrusions 144, 146 extend away from the sleeve portion 116. The protrusions 144, 146 extend substantially longitudinally along at least a portion of the outer surface, between a top edge 148 and the bottom edge 125 of the sleeve portion 116. In some embodiments, the protrusions 144, 146 may extend substantially longitudinally between the top edge 148 and the bottom edge 125.

It is contemplated that, in alternative embodiments, the endoscope component 112 may include the guide 136 while the coolant component 102 may include the pair of protrusions 144, 146 defining the slot 138 to receive the guide 136.

The guide 136 and the slot 138 are geometrically complementary to one another. In this embodiment, the guide 136 has a dovetail shaped profile and the slot 138 has a complementary dovetail shaped profile. An outer face 148 of the guide 136 is curved such that the outer face 148 is flush with the outer surface of the sleeve portion 116 when the guide 136 is received by the slot 138. It is contemplated that, in alternative embodiments, the guide 136 and slot 138 may have a different geometric profile which are complementary to one another.

In some embodiments, the guide 136 may further include a snap clip (not shown) disposed at a bottom portion of the guide 136 to retain the guide 136 within the slot 138, thereby mitigating risk of unintentional uncoupling of the coolant component 102 and the endoscope component 112. In alternative embodiments, the snap clip may be omitted or be replaced by another type of securing mechanism.

It is to be appreciated that different configurations of the first and second engaging members 136, 138 may be used to removably connect the coolant component 102 and the endoscope component 112.

It is contemplated that, in alternative embodiments, the endoscope component 112 may be integrated with a portion of the endoscope 10. For example, in some instances, the portion of the endoscope 10 in which the endoscope component 112 is integrated, may include the protrusions 144, 146 defining the slot 138 for receiving the guide 136, thereby coupling the coolant component 102 to the endoscope 10. Alternatively, the portion of the endoscope 10 in which the endoscope component 112 is integrated may include the guide 136, while the coolant component 102 may include the protrusions 114, 146 defining the slot 138 for receiving the guide 136, thereby coupling the coolant component 102 to the endoscope 10.

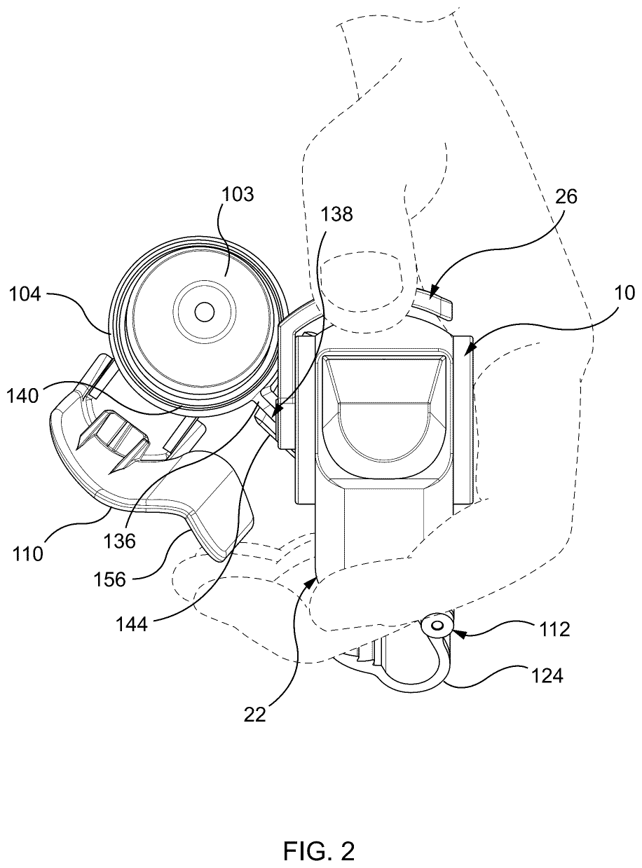

When the coolant component 102 is connected to the endoscope component 112, the coolant component 102 extends rearwardly from the channel opening 122 of the endoscope component 112. As depicted in FIG. 2, the guide 136 is disposed generally parallel to the position of the lever 110 on the coolant component body 104. The protrusions 144, 146 are positioned rearwardly from the channel opening 122. When the coolant component 102 is connected to the endoscope component 112, that is when the guide 136 is inserted into the slot 138, the coolant component 102 is positioned rearwardly from the channel opening 122 of the endoscope component 112. As a result, when the coolant delivery device 100 is mounted to the endoscope 10, the lever 110 is angled such that the grip portion 156 of the lever 110 can be easily reached by fingers of the user and the force applied to the lever 110 is directed inward (i.e., toward a palm of the user). That is, the grip portion 156 of the lever 110 is positioned towards a front end 22 of the endoscope 10, thereby allowing the user to easily reach the grip portion 156 and actuate the lever 110 via a gripping motion with their hand (as depicted in FIG. 20). In other words, the lever 110 is disposed forwardly of the coolant component body 104 and is configured to be brought closer to the coolant component body 104 when it is pulled by the user's fingers. In this embodiment, the user's thumb rests on a back end 26 of the endoscope. With reference to FIG. 3, a longitudinal plane 30 of the coolant component 102 goes through a central axis of the coolant component 102, bisecting the coolant component body 104, the lever 110, and the pivot end 150 of the lever 110. A longitudinal plane 40 of the endoscope component 104 goes through a central axis of the endoscope component 102, bisecting the endoscope component 102 and the handle 12 of the endoscope 10. In this embodiment, the longitudinal plane 30 of the coolant component 102 is not parallel with the longitudinal plane 40 of the endoscope component 104. Specifically, an angle (denoted by θ) formed between the longitudinal plane 30 of the coolant component 102 and the longitudinal plane 40 of the endoscope component 104 is about 30° to 60°. The grip portion 156 of the lever 110 is positioned closer to the front of the endoscope 10, thereby closer to fingers of the user for actuation of the lever 110. The length of the lever 110 and positioning of the grip portion 156 is configured such that a reasonable force applied by the fingers of the user to the end of the lever 110 can actuate the lever 110, releasing the valve and dispensing coolant. It is contemplated that, in alternative embodiments, the angle θ may be about 90°

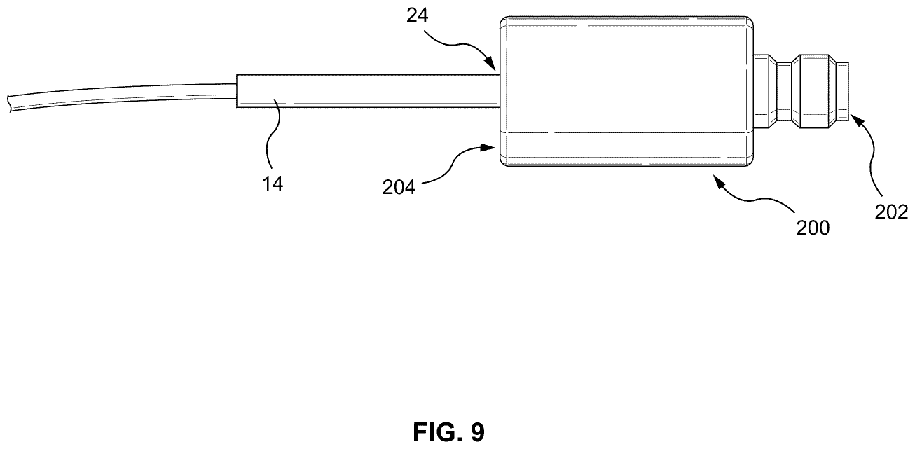

With reference to FIGS. 9 to 13, the exhaust hub 200 will now be described in further detail. The exhaust hub 200 is disposed at a proximal end 24 of the catheter 14. The exhaust hub 200 is connectable to the outlet 106 of the coolant component body 104 to fluidly connect the catheter 14 and the outlet 106. In this embodiment, the exhaust hub 200 has a connection end 202 configured to be inserted into the quick-connect mechanism 108 and a catheter end 204 configured to receive the proximal end 24 of the catheter 14.

As depicted in FIG. 12, the exhaust hub 200 defines a coolant channel 206 extending between the connection end 202 and the catheter end 204. The coolant channel 206 forms the fluid path between the catheter 14 and the outlet 106 such that, upon actuation of the lever 110, coolant flows from the coolant source 12 into the catheter 14.

The exhaust hub 200 further defines a plurality of exhaust channels 208. The exhaust channels 208 are in fluid communication with the catheter 14 for expelling exhaust created during the vaporization of coolant at the distal tip of the catheter 14. The exhaust hub 200 includes internal skives 210 which allow for the exit of the exhaust into the exhaust channels 208. The exhaust channels 208 expel the exhaust away from the coolant delivery device 100 and the user. In other words, the exhaust channels 208 expel exhaust towards the catheter 14. In this embodiment, the exhaust channels 208 are disposed radially around the coolant fluid channel 204. The exhaust hub 200 provides multiple exhaust channels 208 disposed radially around the coolant channel 206, thereby mitigating risk of unintentional blockage of the exhaust, for example by the user's fingers.

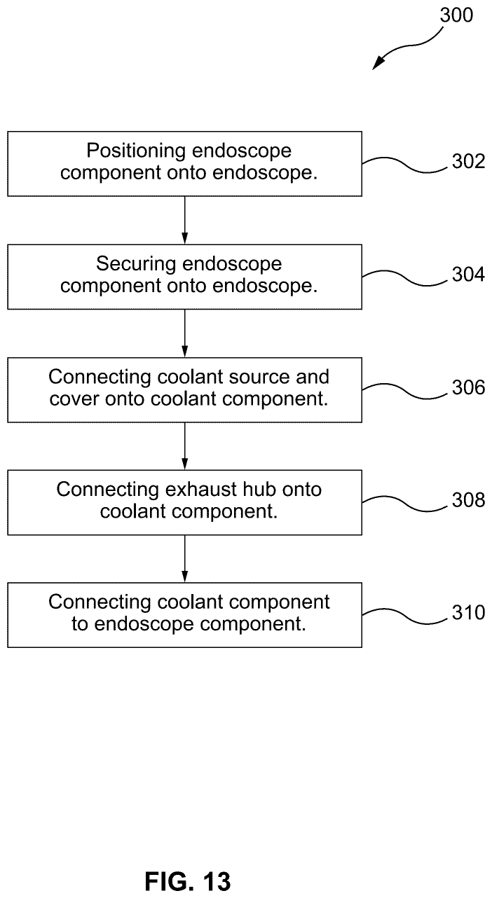

With reference to FIGS. 13 to 20, a method 300 of assembling and using the coolant delivery device 100 will now be described. It is appreciated that the order in which the steps are described are exemplary and may be re-ordered in other embodiments.

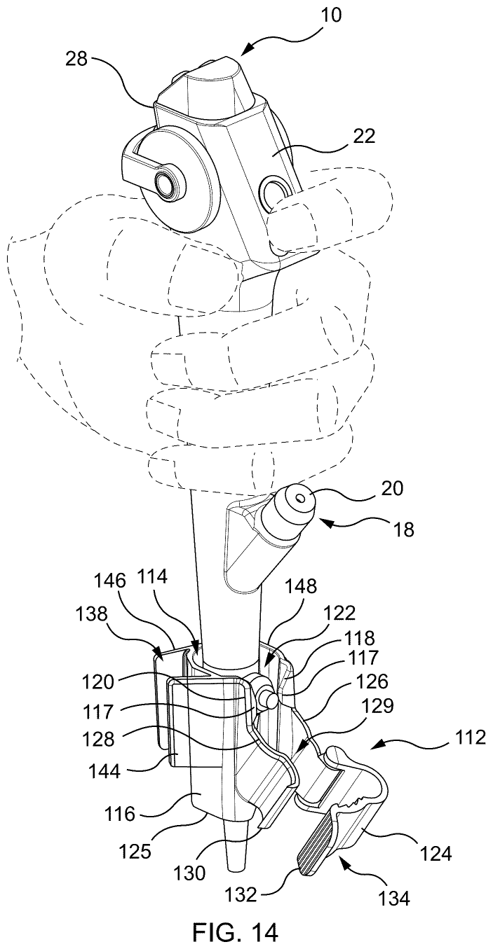

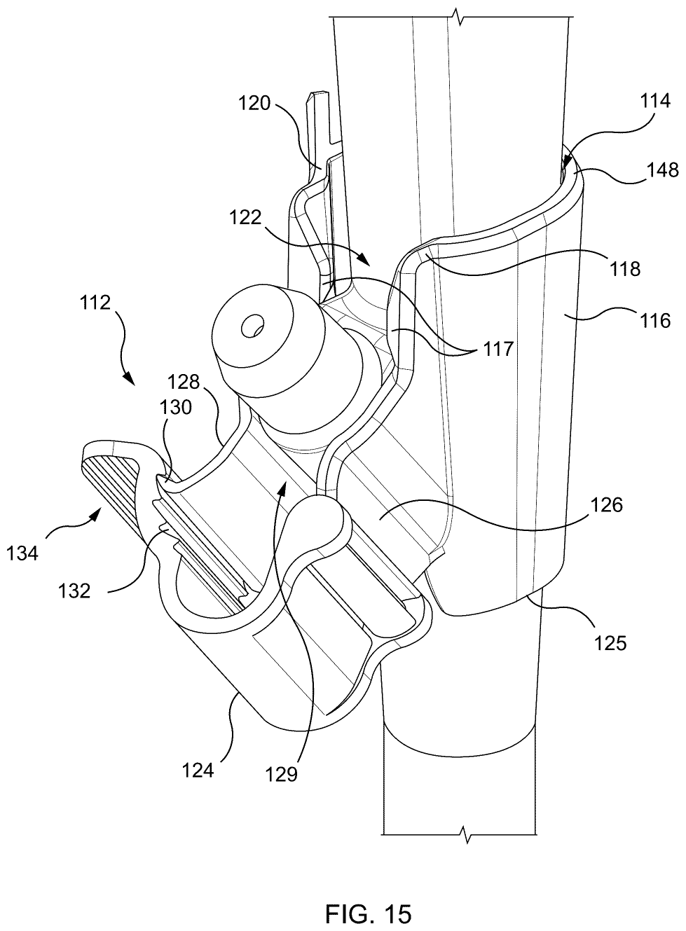

The method 300 begins, at step 302, with positioning the endoscope component 112 onto the lower portion 18 of the endoscope 10. The user may slide the endoscope component 112 from the back end 26 of the endoscope 10 forwardly. As depicted in FIG. 14, the endoscope component 112 may be slid onto the endoscope 10 at a position lower than the final position of the endoscope component 112 on the endoscope 10. The user moves the endoscope component 112 upwards onto the endoscope 10 until the endoscope component 112 is snug around the lower portion 18 of the endoscope 10. In some embodiments, the user may first align the endoscope component 112 with the final position on the lower portion 18 of the endoscope 10 and then slide the endoscope component 112 from the back end 26 of the endoscope 10 forwardly. In this instance, as the endoscope 10 slides into the slot 129 and the channel 114, the endoscope component 112 resiliently flexes around the endoscope 10 before returning to the original shape once the endoscope 10 is fully received.

The method 300 continues, at step 304, with closing the clip 124 (as depicted in FIG. 15), securing the endoscope component 112 onto the endoscope 10. As described above, the tension of the endoscope component 112 around the endoscope 10 may be adjusted by positioning the ridge 130 of the endoscope component 112 into the respective notch 132.

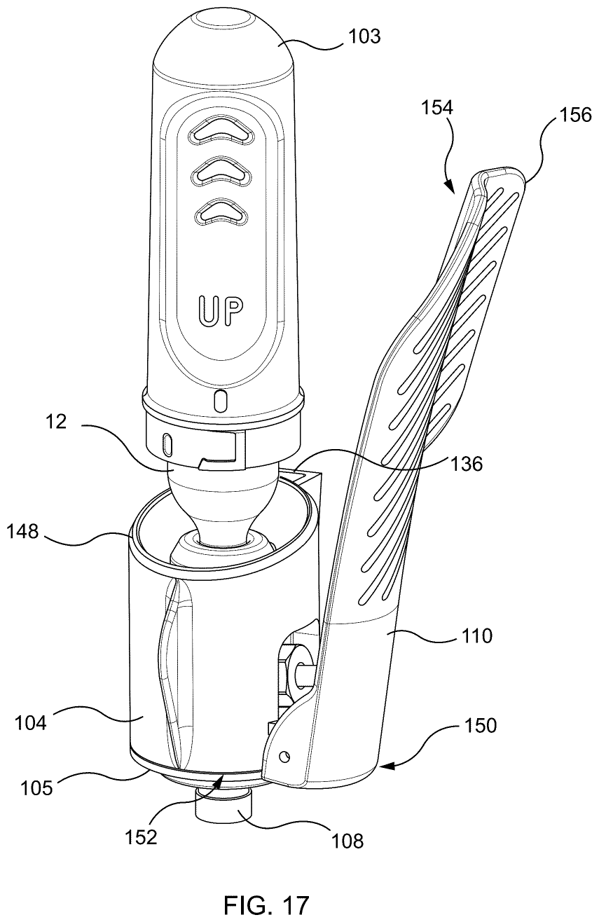

The method 300 continues, at step 306, with connecting the coolant source 12 with the coolant component body 104 (as depicted in FIG. 16) and positioning the cover 103 overtop the coolant source 12 (as depicted in FIG. 17). The coolant source 12 12 is inserted into the coolant component body 104 and twisted into a secured position. The cover 103 is then positioned over the coolant source 12 and connected to the coolant component body 104 via a threaded interface.

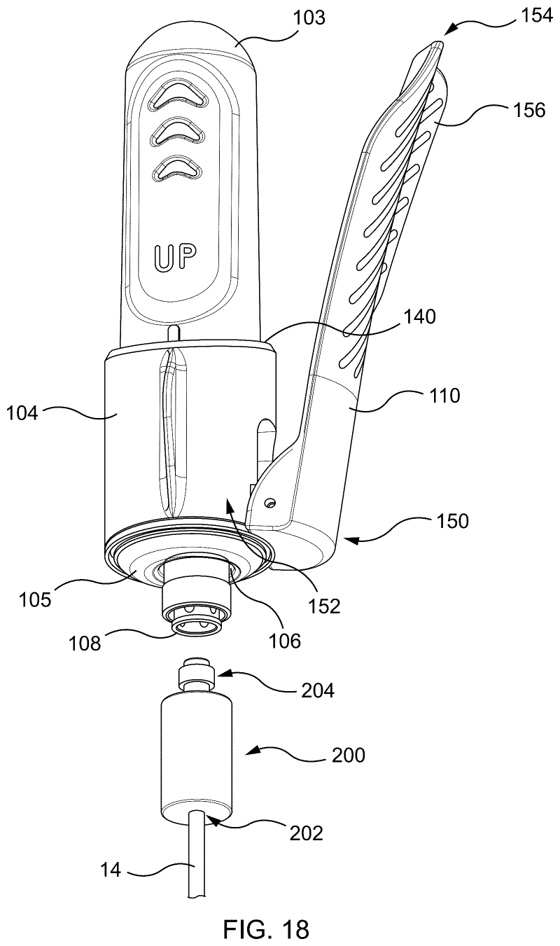

The method 300 continues, at step 308, with connecting the exhaust hub 200 to the coolant component 102. As depicted in FIG. 18, the exhaust hub 200 is connected to the outlet 106 via the quick-connect mechanism 108, thereby fluidly connecting the coolant source 12 with the catheter 14 and providing a pathway for the exhaust generated during vaporization of the coolant.

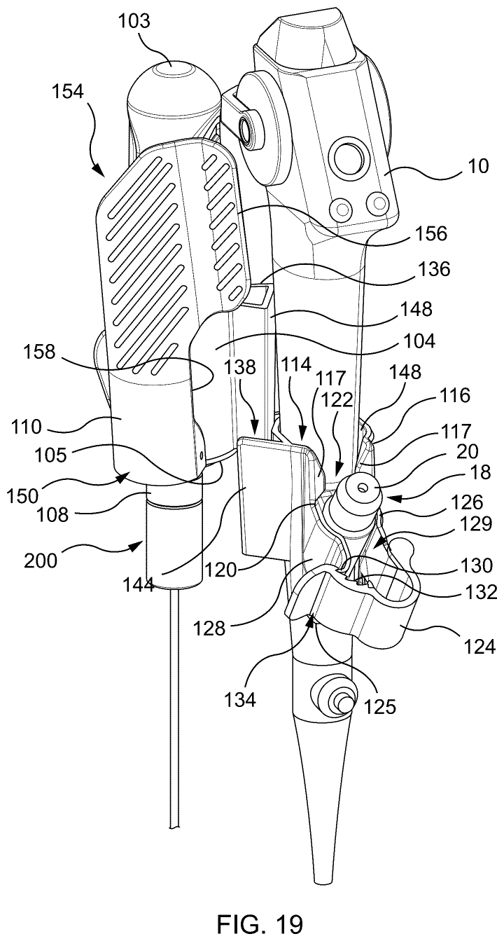

At step 310, the coolant component 102 is connected to the endoscope component 112. As depicted in FIG. 19, the guide 136 disposed on the coolant component body 104 is slidably inserted into the slot 138 of the endoscope component 112. In some instances, the guide 136 includes the snap clip (not shown) to retain the guide 136 within the slot 138 and prevent unintentional uncoupling of the coolant component 102 and the endoscope component 112. It is to be understood that, in some embodiments, the user may desire to keep the coolant component 102 and the endoscope component 112 separate, such that the user controls the endoscope 10 with one hand and the coolant delivery device 100 with the other, and thus this step may be omitted.

As depicted in FIG. 20, once secured, the coolant delivery device 100 is now attached onto the endoscope 10 such that the user may operate the endoscope 10 and the coolant delivery device 100 by a single hand. To control the flow of coolant, the user positions their fingers on the grip portion 156 of the lever 110 and performs a gripping motion, bringing the lever 110 towards the coolant component body 104. To manipulate the endoscope 10, the user may use the thumb of the same hand to actuate the control handle of the endoscope 10.

The disclosed embodiments of the coolant delivery device 100 provide various benefits including, but not limited to, the reduction of cost of capital equipment (i.e., pressurized tanks and control units) for the coolant delivery device 100. Providing users with a disposable cartridge 12 filled with pressurized coolant and the coolant delivery device 100 providing a hand-held actuation for delivery of said coolant (i.e., the lever 110), allows for optimization of space within a surgery room as there is no need for the pressurized tanks and a separate control unit for dispensing the coolant. Additionally, the coolant delivery device 100 and disposable cartridge 12 allow for the system to be easily portable into and out of operating rooms. The attachability of the coolant delivery device 100 to the endoscope 10 enables users to control the endoscope 10 and delivery coolant with a single hand. Further, the design and positioning of the lever 110 on the coolant delivery device 100 is such that only a reasonable amount of force is required by the user to actuate the lever 110, thereby providing users with a comfortable and ergonomic means to actuate the lever 110. That is, users position their fingers on the grip portion 156 and perform a gripping motion to move the lever 110 from an unactuated state to the actuated state, dispensing the coolant. The coolant delivery device 100 is modular to enable flexibility of use. For example, if a user wants to control flow of the coolant with one hand and the endoscope 10 with the other, the user may easily separate the coolant component 102 from the endoscope component 112 by withdrawing the guide 136 of the coolant component 102 from the slot 138 of the endoscope component 112, without the need for any additional removal steps and/or hardware. Similarly, the user may easily connect the coolant component 102 with the endoscope component 112 by sliding the guide 136 of the coolant component 102 into the slot 138 of the endoscope component 112 without any additional installation steps and/or hardware. The endoscope component 112 is configured to adapt to various endoscopes 10 by providing users with a means to adjust the tension of the endoscope component 112 around the endoscope 10 via the securing mechanism 124.

In view of the various disclosures related to the coolant delivery device 100, it will be understood that, although the embodiments presented herein have been described with reference to specific features and structures, it is clear that various modifications and combinations may be made without departing from such disclosures. The specifications and drawings are, accordingly, to be regarded simply as an illustration of the discussed implementations or embodiments and their principles as defined by the appended claims, and are contemplated to cover any and all modifications, variations, combinations, or equivalents that fall within the scope of the present disclosure.

What is claimed is:

1. A coolant delivery device configured to be attachable to an endoscope, the coolant delivery device comprising:
a coolant component having:
a coolant component body configured to removably connect to a coolant source, the coolant source containing a coolant, the coolant component body defining a fluid path from the coolant source to an outlet of the coolant component body; and
an actuation mechanism operatively coupled to the coolant component body and configured to control flow of the coolant from the coolant source; and
an attachment mechanism configured to removably attach the coolant component to the endoscope.

2. The coolant delivery device of claim 1, wherein the attachment mechanism is an endoscope component connected to the coolant component body, the endoscope component defining a channel for selectively receiving a portion of the endoscope.

3. The coolant delivery device of claim 2, wherein the endoscope component comprises a sleeve portion configured to wrap at least partially around the portion of the endoscope, the sleeve portion defining the channel in which the portion of the endoscope can be received to extend through open ends of the sleeve portion, the sleeve portion being longitudinally split to define a channel opening through which the portion of the endoscope will be received and removed.

4. The coolant delivery device of claim 3, further comprising a securing mechanism operatively connected to the endoscope component and configured to modulate a width of the channel opening for adjustably securing the endoscope component to the endoscope.

5. The coolant delivery device of claim 4, wherein the securing mechanism is a clip pivotably connected to the endoscope component, the clip configured to adjust a tension of the attachment of the endoscope component onto the portion of the endoscope.

6. The coolant delivery device of claim 5, the endoscope component further comprises:
a pair of wings, a first of the pair of wings extending from a first edge of the sleeve portion defining the channel opening, and a second of the pair of wings extending from a second edge of the sleeve portion defining the channel opening; and
the clip being pivotably connected to the first of the pair of wings and having a free end configured to connect to the second of the pair of wings at different positions for adjusting the width of the channel opening.

7. The coolant delivery device of claim 3, wherein the coolant component body is disposed on the sleeve portion of the endoscope component.

8. The coolant delivery device of claim 2, wherein the endoscope component and the coolant component are removably connected to one another.

9. The coolant delivery device of claim 8, wherein:
the coolant component body comprises a first engaging member;
the endoscope component comprises a second engaging member; and
the first engaging member is removably connected to the second engaging member.

10. The coolant delivery device of claim 9, wherein:
the first engaging member is a guide extending away from the coolant component body; and
the second engaging member is a slot, the guide being slidably received in the slot.

11. The coolant delivery device of claim 10, wherein the slot comprises a dovetail-shaped profile and the guide comprises a complementary dovetail-shaped profile.

12. The coolant delivery device of claim 2, wherein:
the channel has an inner surface; and
the endoscope component further comprises a liner disposed on at least a portion of the inner surface of the channel.

13. The coolant delivery device of claim 1, wherein:
the actuation mechanism is a lever pivotably connected to the coolant component body, the lever having:
a pivot end pivotably connected to the coolant component body; and
a free end disposed upwardly of the pivot end when the coolant delivery device is attached to the endoscope.

14. The coolant delivery device of claim 13, wherein the lever is pivotably movable between:
an unactuated position; and
an actuated position in which the lever is pivoted towards the coolant component body to dispense the coolant from the coolant source.

15. The coolant delivery device of claim 13, the lever comprises a grip portion extending from a side edge of the lever.

16. The coolant delivery device of claim 15, wherein the grip portion is disposed at a distal end of the lever.

17. The coolant delivery device of claim 15, wherein:
the lever is pivotably connected to a lower section of the coolant component body; and
the grip portion is disposed upwardly from the lower section of the coolant component body.

18. The coolant delivery device of claim 13, further comprising the endoscope, and wherein:
the endoscope has a front end and a back end,
the front end configured for a user's fingers to rest; and
the back end configured for a user's thumb to rest; and
the lever being disposed towards the front end of the endoscope when the coolant delivery device is attached to the endoscope such that the lever can be actuated by at least one of the user's fingers.

19. A coolant component of a coolant delivery device, the coolant delivery device being configured to be attachable to an endoscope, the coolant component comprising:
a coolant component body configured to removably connect to a coolant source, the coolant source containing a coolant; and
a lever pivotably connected to the coolant component body for controlling flow of the coolant, the lever being selectively pivotable between:
an unactuated position; and
an actuated position in which the lever is pivoted towards the coolant component body,
in response to the lever being in the actuated position, the coolant is dispensed from the coolant source through an outlet of the coolant component body.

20. A coolant delivery device configured to be attachable to an endoscope, the coolant delivery device comprising:
a coolant component having:
a coolant component body configured to removably connect to a coolant source, the coolant source containing a coolant, the coolant component body defining a fluid path from the coolant source to an outlet of the coolant component body; and
an endoscope component connected to the coolant component body, the endoscope component having:
a sleeve portion defining a channel for selectively receiving a portion of the endoscope through open ends of the sleeve portion, the sleeve portion being longitudinally split to define a channel opening through which the portion of the endoscope will be received and removed.
